SEQUENCE LISTING (41 sequences)

SEQ ID NO: 1 - protein, 495 aa, synthetic construct
MWRPSDSTVYVPPPNPVSKVVATDAYVTRTNIFYHASSSRLLAVGHPYFSIKRANKTVVPKVSGYQYRVFKVVLPDPNKFALPDSSLFDPTTQRLVWACTGLEVGRGQPLGVGVSGHPFLNKYDDVENSGSGGNPGQDNRVNVGMDYKQTQLCMVGCAPPLGEHWGKGKQCTNTPVQAGDCPPLELITSVIQDGDMVDTGFGAMNFADLQTNKSDVPIDICGTTCKYPDYLQMAADPYGDRLFFFLRKEQMFARHFFNRAGEVGEPVPDTLIIKGSGNRTSVGSSIYVNTPSGSLVSSEAQLFNKPYWLQKAQGHNNGICWGNQLFVTVVDTTRSTNMTLCASVTTSSTYTNSDYKEYMRHVEEYDLQFIFQLCSITLSAEVMAYIHTMNPSVLEDWNFGLSPPPNGTLEDTYRYVQSQAITCQKPTPEKEKPDPYKNLSFWEVNLKEKFSSELDQYPLGRKFLLQSGYKAKPKLKRAAPTSTRTSSAKRKKVKK

SEQ ID NO: 2 - DNA, 54 bp, synthetic construct
  source 1..54: mol_type = other DNA
tctagtggtggcggtggttcgggcggtggtggaggtggtagttctagatcttcc

SEQ ID NO: 3 - DNA, 741 bp, synthetic construct
  source 1..741: mol_type = other DNA
gagctcgtgatgacccagactccatcctccgtgtctgcagctgtgggaggcacagtcaccatcaattgtcaggccagtcagagtgtttatagtaacaacctagtctggtatcagcagaaaccagggcagcctcccaagctcctgatctatggtgcatccactctggcatctggggtcccatcgcggttcaaaggcagtggatctgggacacagttcactctcaccatcagcgatgtggtgtgtgacgatgctgccacttactactgtggagggtatgttagtgatagtactgatggtactgctttcggcggagggaccgaggtggtcgtcaaatctagtggtggcggtggttcgggcggtggtggaggtggtagttctagatcttcccagcagcagctggaggagtccgggggaggcctggtcaagcctggaggaaccctgacactcacctgtaaagcctctggaatcgacctcagtaactactacatgtgctgggtccgccaggctccagggaaggggctggagtggatcggatgcatttatactggtagtggtcgcacttactacgcgagctgggcgaaaggccgattcaccatctccaaggcctcgtcgaccacggtgactctgcaaatgaccagtctgacagccgcggacacggccacctatttctgtgcgagagattatcttggttggcgtgccagtaacatctggggcccaggtaccctggtcacagtgagctct

SEQ ID NO: 4 - protein, 247 aa, synthetic construct
ELVMTQTPSSVSAAVGGTVTINCQASQSVYSNNLVWYQQKPGQPPKLLIYGASTLASGVPSRFKGSGSGTQFTLTISDVVCDDAATYYCGGYVSDSTDGTAFGGGTEVVVKSSGGGGSGGGGGGSSRSSQQQLEESGGGLVKPGGTLTLTCKASGIDLSNYYMCWVRQAPGKGLEWIGCIYTGSGRTYYASWAKGRFTISKASSTTVTLQMTSLTAADTATYFCARDYLGWRASNIWGPGTLVTVSS

SEQ ID NO: 5 - protein, 12 aa, synthetic construct
QASQSVYSNNLV

SEQ ID NO: 6 - protein, 7 aa, synthetic construct
GASTLAS

SEQ ID NO: 7 - protein, 12 aa, synthetic construct
GGYVSDSTDGTA

SEQ ID NO: 8 - protein, 5 aa, synthetic construct
NYYMC

SEQ ID NO: 9 - protein, 17 aa, synthetic construct
CIYTGSGRTYYASWAKG

SEQ ID NO: 10 - protein, 10 aa, synthetic construct
DYLGWRASNI

SEQ ID NO: 11 - DNA, 354 bp, synthetic construct
  source 1..354: mol_type = other DNA
cagcagcagctggaggagtccgggggaggcctggtcaagcctggaggaaccctgacactcacctgtaaagcctctggaatcgacctcagtaactactacatgtgctgggtccgccaggctccagggaaggggctggagtggatcggatgcatttatactggtagtggtcgcacttactacgcgagctgggcgaaaggccgattcaccatctccaaggcctcgtcgaccacggtgactctgcaaatgaccagtctgacagccgcggacacggccacctatttctgtgcgagagattatcttggttggcgtgccagtaacatctggggcccaggtaccctggtcacagtgagctct

SEQ ID NO: 12 - protein, 118 aa, synthetic construct
QQQLEESGGGLVKPGGTLTLTCKASGIDLSNYYMCWVRQAPGKGLEWIGCIYTGSGRTYYASWAKGRFTISKASSTTVTLQMTSLTAADTATYFCARDYLGWRASNIWGPGTLVTVSS

SEQ ID NO: 13 - DNA, 57 bp, synthetic construct
  source 1..57: mol_type = other DNA
atgggctggtccctgattctgctgttcctggtggctgtggctaccagggtgctgagt

SEQ ID NO: 14 - protein, 19 aa, synthetic construct
MGWSLILLFLVAVATRVLS

SEQ ID NO: 15 - DNA, 972 bp, synthetic construct
  source 1..972: mol_type = other DNA
ggtcaacctaaggctccgtcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaataa

SEQ ID NO: 16 - protein, 323 aa, synthetic construct
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 17 - DNA, 1383 bp, synthetic construct
  source 1..1383: mol_type = other DNA
atgggctggtccctgattctgctgttcctggtggctgtggctaccagggtgctgagtcagcagcagctggaggagtccgggggaggcctggtcaagcctggaggaaccctgacactcacctgtaaagcctctggaatcgacctcagtaactactacatgtgctgggtccgccaggctccagggaaggggctggagtggatcggatgcatttatactggtagtggtcgcacttactacgcgagctgggcgaaaggccgattcaccatctccaaggcctcgtcgaccacggtgactctgcaaatgaccagtctgacagccgcggacacggccacctatttctgtgcgagagattatcttggttggcgtgccagtaacatctggggcccaggtaccctggtcacagtgagctctggtcaacctaaggctccgtcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaataa

SEQ ID NO: 18 - protein, 460 aa, synthetic construct
MGWSLILLFLVAVATRVLSQQQLEESGGGLVKPGGTLTLTCKASGIDLSNYYMCWVRQAPGKGLEWIGCIYTGSGRTYYASWAKGRFTISKASSTTVTLQMTSLTAADTATYFCARDYLGWRASNIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 19 - DNA, 333 bp, synthetic construct
  source 1..333: mol_type = other DNA
gagctcgtgatgacccagactccatcctccgtgtctgcagctgtgggaggcacagtcaccatcaattgtcaggccagtcagagtgtttatagtaacaacctagtctggtatcagcagaaaccagggcagcctcccaagctcctgatctatggtgcatccactctggcatctggggtcccatcgcggttcaaaggcagtggatctgggacacagttcactctcaccatcagcgatgtggtgtgtgacgatgctgccacttactactgtggagggtatgttagtgatagtactgatggtactgctttcggcggagggaccgaggtggtcgtcaaa

SEQ ID NO: 20 - protein, 111 aa, synthetic construct
ELVMTQTPSSVSAAVGGTVTINCQASQSVYSNNLVWYQQKPGQPPKLLIYGASTLASGVPSRFKGSGSGTQFTLTISDVVCDDAATYYCGGYVSDSTDGTAFGGGTEVVVK

SEQ ID NO: 21 - DNA, 57 bp, synthetic construct
  source 1..57: mol_type = other DNA
atgggctggtcctgtatcatcctgttcctggtggctacagccacaggagtgcatagt

SEQ ID NO: 22 - protein, 19 aa, synthetic construct
MGWSCIILFLVATATGVHS

SEQ ID NO: 23 - DNA, 315 bp, synthetic construct
  source 1..315: mol_type = other DNA
ggggatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttaa

SEQ ID NO: 24 - protein, 104 aa, synthetic construct
GDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 25 - DNA, 705 bp, synthetic construct
  source 1..705: mol_type = other DNA
atgggctggtcctgtatcatcctgttcctggtggctacagccacaggagtgcatagtgagctcgtgatgacccagactccatcctccgtgtctgcagctgtgggaggcacagtcaccatcaattgtcaggccagtcagagtgtttatagtaacaacctagtctggtatcagcagaaaccagggcagcctcccaagctcctgatctatggtgcatccactctggcatctggggtcccatcgcggttcaaaggcagtggatctgggacacagttcactctcaccatcagcgatgtggtgtgtgacgatgctgccacttactactgtggagggtatgttagtgatagtactgatggtactgctttcggcggagggaccgaggtggtcgtcaaaggggatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttaa

SEQ ID NO: 26 - protein, 234 aa, synthetic construct
MGWSCIILFLVATATGVHSELVMTQTPSSVSAAVGGTVTINCQASQSVYSNNLVWYQQKPGQPPKLLIYGASTLASGVPSRFKGSGSGTQFTLTISDVVCDDAATYYCGGYVSDSTDGTAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 27 - DNA, 24 bp, synthetic construct
  source 1..24: mol_type = other DNA
gagctcgtgmtgacccagactcca

SEQ ID NO: 28 - DNA, 24 bp, synthetic construct
  source 1..24: mol_type = other DNA
gagctcgatmtgacccagactcca

SEQ ID NO: 29 - DNA, 24 bp, synthetic construct
  source 1..24: mol_type = other DNA
gagctcgtgatgacccagactgaa

SEQ ID NO: 30 - DNA, 21 bp, synthetic construct
  source 1..21: mol_type = other DNA
taggatctccagctcggtccc

SEQ ID NO: 31 - DNA, 21 bp, synthetic construct
  source 1..21: mol_type = other DNA
tttgatttccacattggtgcc

SEQ ID NO: 32 - DNA, 21 bp, synthetic construct
  source 1..21: mol_type = other DNA
tttgacsaccacctcggtccc

SEQ ID NO: 33 - DNA, 21 bp, synthetic construct
  source 1..21: mol_type = other DNA
cagtcggtggaggagtccrgg

SEQ ID NO: 34 - DNA, 21 bp, synthetic construct
  source 1..21: mol_type = other DNA
cagtcggtgaaggagtccgag

SEQ ID NO: 35 - DNA, 21 bp, synthetic construct
  source 1..21: mol_type = other DNA
cagtcgytggaggagtccggg

SEQ ID NO: 36 - DNA, 23 bp, synthetic construct
  source 1..23: mol_type = other DNA
cagsagcagctgrtggagtccgg

SEQ ID NO: 37 - DNA, 24 bp, synthetic construct
  source 1..24: mol_type = other DNA
tgargagayggtgaccagggtgcc

SEQ ID NO: 38 - DNA, 33 bp, synthetic construct
  source 1..33: mol_type = other DNA
accagggtgctgagtcagcagcagctggaggag

SEQ ID NO: 39 - DNA, 24 bp, synthetic construct
  source 1..24: mol_type = other DNA
tgtgaccagggtacctgggcccca

SEQ ID NO: 40 - DNA, 35 bp, synthetic construct
  source 1..35: mol_type = other DNA
acaggagtgcatagtgagctcgtgatgacccagac

SEQ ID NO: 41 - DNA, 35 bp, synthetic construct
  source 1..35: mol_type = other DNA
ggtgcaactggatcccctttgacgaccacctcggt

(19) United States
(12) Patent Application Publication
(10) Pub. No.: US 2024/0158474 A1
(43) Pub. Date: May 16, 2024

(54) ANTI-HPV6 L1 PROTEIN ANTIBODY AND DETECTION METHOD USING THE ANTIBODY

(71) Applicant: Sino Biological, Inc., Beijing (CN)

(72) Inventors: Liangzhi XIE, Beijing (CN); Jie ZHANG, Beijing (CN); Wei REN, Beijing (CN); Xuman WANG, Beijing (CN); Yan LI, Beijing (CN); Ran GAO, Beijing (CN)

(21) Appl. No.: 18/469,055

(22) Filed: Sep. 18, 2023

(30) Foreign Application Priority Data
Nov. 15, 2022 (CN) 202211426888.6

(51) Int. Cl. C07K 16/08; G01N 33/569

(52) U.S. Cl. CPC C07K 16/084; G01N 33/56983; C07K 2317/565

(57) ABSTRACT

The disclosure relates to the field of molecular virology and immunology, and discloses an anti-HPV6 L1 protein antibody and a detection method using the antibody. The antibody or its antigen-binding fragment comprises a light chain variable region or part thereof and/or a heavy chain variable region or part thereof, the light chain variable region or part thereof comprises one or more of the light chain CDR1-3 whose amino acid sequence is SEQ ID NOs: 5-7 respectively; the heavy chain variable region or part thereof comprises one or more of heavy chain CDR1-3 whose amino acid sequence is SEQ ID NOs: 8-10. The antibody has strong binding ability to HPV6L1 antigen, and has no cross-reaction with HPV11, 16, 18, 31, 33, 35, 39, 45, 51, 52, 56, 58 and 59 types, and is suitable for performing immunogenicity evaluation of HPV vaccine as a detection antibody in ELISA quantification.

### CROSS REFERENCE TO RELATED APPLICATIONS

This application claims priority to Chinese Application No. 202211426888.6, filed on Nov. 15, 2022, entitled “anti-HPV6 L1 protein antibody and detection method using the antibody”, which is specifically and entirely incorporated by reference.

### SEQUENCE LISTING

The Instant Application contains a Sequence Listing which has been submitted electronically in XML format and is hereby incorporated by reference in its entirety. Said XML copy, created on Sep. 7, 2023 is named “IUS84830YQSZ.xml” and is 43,669 bytes in size. The Sequence Listing does not go beyond the disclosure in the application as filed.

### FIELD

The disclosure belong to that field of molecular virology and immunology, in particular to antibodies or antigen-binding fragment thereof capable of specifically binding human papillomavirus (HPV6) L1 protein, nucleic acids encoding the same, vectors, cell, use of the antibodies or antigen-binding fragments thereof in detecting HPV6 L1 protein, and methods for detecting or quantitatively determining HPV6 L1 protein using the antibodies or antigen-binding fragments thereof.

### BACKGROUND

Human papilloma virus (HPV) is an epitheliophilic, highly tissue-specific, non-enveloped DNA virus. The late region of its DNA (L) open reading frame encodes a major capsid protein L1 and a minor capsid protein L2; the high variability of L2 protein is related to the polymorphism of virus antigen. L1 protein, with high conservation, is the main type-specific antigen that can induce the body to produce neutralizing antibodies, which are often used as antigens for HPV vaccines. More than 100 types of HPV have been identified and different subtypes of HPV have different tissue preferences and trigger diseases ranging from benign warts to epithelial cell tumors (including the cervix, vagina, vulva, anus and throat).

Cervical cancer ranks the second in female cancer mortality worldwide and has a younger onset trend. Fifteen high-risk (HR)HPV types can lead to the cervix, anus, penis, vagina, vulva, and oropharyngeal cancer. Of these, HPV-16 and HPV-18 are by far the most common causes of cancer, accounting for approximately 70% of cervical cancer, with the remainder being caused by other HR-HPV types (31, 33, 35, 39, 45, 51, 52, 56, 58, 59, 68, 73 and 82). HPV-16 accounts for approximately 95% of HPV-positive oropharyngeal cancer (OPCs). Persistent low-risk genotypes of HPV-6 and HPV-11 contribute to most anogenital warts and respiratory papillomas, but are rarely associated with cancer (Human Papilloma Virus in Clinical Cancer and Oropharyngeal Cancer: One Cause, Two Diseases Tara A. Berman and John T. Schiller, PhD 2 Cancer 2017; 123:2219-29).

Cervical cancer vaccine is the demand of human health. The L1 protein, which can self-assemble into virus-like particles (VLP) (Structure of Small Virus-like particles assembled from the L1 protein of Human Papilloma Virus 16 Chen, X. S., R. L. Garcea, mol.Cell.5 (3): 557-567, 2000), seems to be an excellent candidate for a papillomavirus vaccine. VLP is morphologically similar to a true virosome and is capable of inducing highly valuable neutralizing antibodies after administration to animals or humans. Because VLPs do not contain a potentially carcinogenic viral genome, they have safely replaced live viruses in HPV vaccine development (see Schiller and Hidesheim, J. Clin. Virol.19: 67-74 (2000) for a review).

Bivalent recombinant HPV vaccine CERVARIX® manufactured by Glaxo (https://www.fda.gov/Downloads/BiologicsBloodVaccines/Vaccines/Approved Products/UCM186981.pdf) as well as the 4-valent recombinant HPV vaccine GARDASIL®-4 produced by Merck (https://www.fda.gov/vaccinees-blood-biologics/vaccines/Gardasil) and the 9-valent recombinant HPV vaccine GARDASIL®-9 recombination vaccines (https://www.fda.gov/vaccinees-blood-biologics/vaccines/Gardasil-9) are VLP vaccines.

Given the high incidence and mortality of cervical cancer, there is still a need to develop more low-cost, safe and efficient novel HPV VLP vaccines. HPV virus-like particles have large molecular weight and complex structure, and whether the integrity and the structure of the HPV virus-like particles are correct or not can directly affect the immune and protection effects, so the field still needs specific neutralizing antibodies aiming at specific HPV types and even HPV VLP with specific integrity and the structure being correct, and the HPV virus-like particles can be sensitively and highly specifically detected, so as to better evaluate the produced vaccine and the protection effects thereof.

### SUMMARY

The purpose of the present disclosure to provide a specific antibody against specific HPV types, even against a specific HPV VLP with specific integrity and correct structural, to provide an anti-HPV6 L1 protein antibody or antigen binding fragment thereof with high specificity.

The first aspect of the disclosure relates to an anti-HPV6 L1 protein antibody or antigen-binding fragment thereof comprising a light chain variable region or part thereof and/or a heavy chain variable region or part thereof,
wherein the light chain variable region or part thereof comprises one or more of a light chain CDR1 having an amino acid sequence of SEQ ID NO:5, a light chain CDR2 having an amino acid sequence of SEQ ID NO:6, and a light chain CDR3 having an amino acid sequence of SEQ ID NO:7; and
the heavy chain variable region or part thereof comprises one or more of a heavy chain CDR1 having an amino acid sequence of SEQ ID NO:8, a heavy chain CDR2 having an amino acid sequence of SEQ ID NO:9, and a heavy chain CDR3 having an amino acid sequence of SEQ ID NO:10.

In one embodiment, the antibody or antigen-binding fragment thereof comprises an amino acid sequence having at least 90%, 92%, 95%, 98% or 100% sequence identity to the amino acid sequence SEQ ID NO:20 of the light chain variable region of an anti-HPV6 L1 protein antibody, and/or an amino acid sequence have at least 90%, 92%, 95%, 98% or 100% sequence identity to the amino acid sequence SEQ ID NO:12 of the heavy chain variable region of an anti-HPV6 L1 protein antibody.

In one embodiment, the antibody further comprises a light chain constant region and a heavy chain constant region. In a preferred embodiment, the light chain constant region is an amino acid sequence have at least 90%, 92%, 95%, 98% or 100% sequence identity to the amino acid sequence SEQ ID NO:24 of the light chain constant region of an anti-HPV6 L1 protein antibody, and/or the heavy chain constant region is an amino acid sequence have at least 90%, 92%, 95%, 98% or 100% sequence identity to the amino acid sequence SEQ ID NO:16 of the heavy chain constant region of an anti-HPV6 L1 protein antibody.

In one embodiment, the antibody is a monoclonal antibody.

In one embodiment, the antibody specifically binds to HPV6 L1 antigen without cross-reactivity with other types of HPV L1 antigen; Preferably, that other type of HPV are HPV types 11, 16, 18, 31, 33, 35, 39, 45, 51, 52, 56, 58 and 59.

In one embodiment, the antigen binding fragment is in the form of Fv, Fab, Fab′, Fab′-SH, F(ab′)2, Fd fragment, Fd′ fragment, single chain antibody molecule, or single domain antibody; Among them, the single-chain antibody molecule is preferably an scFv, a di-scFv, a tri-scFv, a bispecific antibody, or an scFab.

The antibody may be an isolated antibody.

The second aspect of the present disclosure relates to a nucleic acid comprising: a nucleotide sequence encoding an anti-HPV6 L1 protein antibody or an antigen-binding fragment thereof according to the first aspect. The nucleic acid may be an isolated nucleic acid.

The third aspect of the disclosure relates to a vector comprising the nucleic acid of the second aspect.

The fourth aspect of the present disclosure relates to a cell comprising the nucleic acid of the second aspect, and/or the vector of the third aspect. The cells may be isolated cells.

The fifth aspect of the disclosure relates to the use of the antibody or antigen-binding fragment thereof according to the first aspect in the detection of HPV6 L1 protein.

The sixth aspect of the disclosure relates to a method for detecting the presence of HPV6 L1 protein in a sample comprising:
a) coating the anti-HPV6 L1 protein antibody or antigen-binding fragment thereof according to the first aspect onto an assay surface;
b) contacting said assay surface with the sample to be tested for a period of time sufficient to allow binding, and then washing said assay surface;
c) contacting the assay surface with the anti-HPV6 L1 protein antibody or antigen-binding fragment thereof of the first aspect conjugated to a reporter group for a period of time sufficient to allow binding of the conjugated antibody, and then washing said assay surface; and
d) detecting the signal of the reporter group.

The seventh aspect of the present disclosure relates to a method for quantitative determination of HPV6 L1 protein comprising:
a) coating the anti-HPV6 L1 protein antibody or antigen-binding fragment thereof according to the first aspect onto an assay surface;
b) contacting said assay surface with the sample to be tested for a period of time sufficient to allow binding, and then washing said assay surface;
c) contacting said assay surface with the anti-HPV6 L1 protein antibody or an antigen-binding fragment thereof of the first aspect conjugated to a reporter group for a period of time sufficient to allow binding of the conjugated antibody, and then washing said assay surface; and
d) quantifying the HPV6 L1 protein by comparison to a standard curve.

In one embodiment, that standard curve is establish through the following steps:
a) coating an anti-HPV6 L1 protein antibody or antigen-binding fragment thereof according to the first aspect onto an assay surface;
b) contacting said assay surface with a series of concentrations of HPV6-L1 protein standard for a period of time sufficient to allow binding, and then washing said assay surface;
c) contacting said assay surface with the anti-HPV6 L1 protein antibody or an antigen-binding fragment thereof of the first aspect conjugated to a reporter group for a period of time sufficient to allow binding of the conjugated antibody, and then washing said assay surface, and measure that absorbance thereof; and
d) drawing a standard curve with the concentration of the standard substance as the vertical coordinate and the corresponding absorbance as the horizontal coordinate.

Wherein the antibody or antigen-binding fragment thereof according to the first aspect of steps (a) and (c) may be the same or different.

In one embodiment, wherein the reporter group is horseradish peroxidase.

In the present disclosure, means and methods existing in the art are employed unless otherwise specified.

Compared with the prior art, the anti-HPV6 L1 protein antibody screened by the disclosure and the antigen binding fragment thereof have significant technical effects; in particular, the antibody and the antigen binding fragment thereof have strong binding force with the HPV6 L1 antigen and high specificity, and do not recognize HPV types 11, 16, 18, 31, 33, 35, 39, 45, 51, 52, 56, 58 and 59. It did not cross-react with the above mixture of type 13 at 50 ng/mL and was suitable for immunogenicity evaluation of HPV vaccine as a detection antibody in ELISA quantitation.

### BRIEF DESCRIPTION OF DRAWINGS

The FIGURE shows the HPV6 VLP quantitation standard curve.

### DETAILED DESCRIPTION

### Definition

Unless otherwise specified, all technical and scientific terms used herein have the meaning commonly understood by one of ordinary skill in the art to which this disclosure belongs. To facilitate an understanding of the present disclosure, the following general meanings of the terms are cited.

The term “antibody” means an immunoglobulin molecule, and refers to any form of antibody that exhibits the desired biological activity. Include, but are not limit to, monoclonal antibodies (include full-length monoclonal antibodies), polyclonal antibodies and multispecific antibodies (e.g., bispecific antibody), and even antibody fragments. Typically, the full-length antibody structure preferably comprises four polypeptide chains, usually two heavy (H) and two light (L) chains interconnected by disulfide bonds. Each heavy chain comprises a heavy chain variable region and a heavy chain constant region. Each light chain comprises a light chain variable region and a light chain constant region. In addition to this typical full-length antibody structure, its structure includes other derived form.

The term “complementarity determining region” (CDRs, e.g., CDR1, CDR2, and CDR3) refers to those amino acid residues of the variable region of an antibody whose presence is necessary for antigen binding. Each variable region typically has three CDR regions identified as CDR1, CDR2, and CDR3. Each complementarity determining region may comprise amino acid residues from a “complementarity determining region” as defined by Kabat (Kabat et al., sequences of proteins of immunological interest, 5th ed. public health service, National Institutes of Health, Bethesda, M D. 1991) and/or those residues from the “hypervariable ring” (Chothia and Lesk; J Mol Biol 196: 901-917 (1987)).

Each of the heavy chain variable region and the light chain variable region typically contains 3 CDRs and up to 4 FRs arranged in the following order, for example, from the amino terminal to the carboxy terminal: FR1, CDR1, FR2, CDR2, FR3, CDR3, FR4.

The complementarity determining region (CDR) of a given antibody may be identified using the Kabat system (Kabat et al., Sequences of Proteins of Immunological Interest, the fifth edition, U.S. Department of Health and Human Services, PHS, NIH, NIH Publication No. 91-3242, 1991).

“Antigen-binding fragment of an antibody” comprises a portion of an intact antibody molecule, retains at least some of the binding specificity of a parent antibody and typically includes at least a portion of an antigen-binding region or variable region (e.g., one or more CDRs) of the parent antibody. Examples of antigen-binding fragments include, but are not limited to, Fv, Fab, Fab′, Fab′-SH, F(ab′)2, Fd fragments, Fd′ fragments, single-chain antibody molecules (e.g., scFv, di-scFv or tri-scFv, bispecific antibodies or scFab), single-domain antibodies.

“Monoclonal antibody” refers to an antibody obtained from a substantially homogeneous antibody population, i.e., the population comprising a single antibody is identical except for possible mutations (e.g., natural mutations) that may be present in minimal amounts. Thus, the term “monoclonal” indicates the nature of the antibody, i.e., not a mixture of unrelated antibodies. In contrast to polyclonal antibody formulations that typically include different antibodies directed to different determinants (epitopes), each monoclonal antibody of a monoclonal antibody formulation is directed to a separate determinant on the antigen. In addition to their specificity, monoclonal antibody preparations have the advantage that they are generally not contaminated with other antibodies. The term “monoclonal” cannot be understood as requiring the production of the antibody by any particular method. The term monoclonal antibody specifically includes chimeric antibodies, humanized antibody and human antibody.

An antibody “specifically binds” to an antigen of interest, such as a tumor-associated polypeptide antigen target (herein, HPV6 VLP protein), i.e., binds to the antigen with sufficient affinity so that the antibody can be used as a therapeutic agent, targets cells or tissues expressing the antigen, and does not significantly cross-react with other protein or with protein other than the homologues and variants (e.g., mutant forms, splice variants, or proteolytic truncated forms) of the antigen target mentioned above.

The term “epitope” includes any protein determinant capable of specifically binding to an antibody or t cell receptor. Epitope determinants generally consist of chemically active surface groups of a molecule (e.g., amino acids or sugar side chains, or combinations thereof) and generally have specific three-dimensional structural characteristics as well as specific charge characteristics.

An “isolated” antibody is an antibody that has been identified and isolated from a component of a cell expressing it. The contaminating components of the cells are substances that interfere with the diagnostic or therapeutic use of the antibodies and may include enzymes, hormones and other solutes of protein or non-protein nature. The isolated natural antibody comprises an in situ antibody within a recombinant cell because at least one component of the natural environment of the antibody is absent. Typically, however, the isolated antibody is prepared by at least one purification step.

The term “HPVL1 protein” as used herein, the terms “HPV” and “human papillomavirus” refer to an uncoated double-stranded DNA virus of the family Papillomaviridae. Their genomes are round and about 8,000 base pairs in size. Most HPV encodes eight major proteins, six in the “early” region (E1-E2), and two in the “late” region (L1 (major capsid protein) and L2 (minor capsid protein)). Over 120 HPV types have been identified and are labeled with numbers (e.g., HPV-16, HPV-18, etc.).

The term “HPV” or “HPV virus” refers to a papillomavirus of the family Papillomaviridae, which is an uncoated DNA virus having a double-stranded closed-loop DNA genome of about 8 kb in size and which can generally be divided into three regions: \textcircled{1} an early region (E) containing six open reading frames encoding nonstructural proteins related to E1, E2, E4-E7 virus replication, transcription and transformation, as well as E3 and E8 open reading frames; \textcircled{2} the late region (L) contains a reading frame encoding the major capsid protein L1 and the minor capsid protein L2; \textcircled{3} long regulatory region (LCR) does not encode any protein, but it has the origin of replication and multiple transcription factor binding sites.

The terms “HPV L1 protein” and “HPV L2 protein” refer to proteins encoded by the late region (L) of the HPV gene and synthesized late in the HPV infection cycle. L1 protein is the major capsid protein and has a molecular weight of 55-60 kDa. The L2 protein is the minor capsid protein. Seventy-two L1 pentamers formed the shell of icosahedral HPV virus particles and wrapped the closed-loop double-stranded DNA micro-chromosome. The L2 protein was located medial to the L1 protein (Structure of Small virus-like particles assembled from the L1 protein of Human Papilloma virus 16 Chen, X. S., R. L. Garcea, mol. Cell.5 (3): 557-567, 2000).

The term “HPV VLP protein” refer to that when the L1 protein is recombinant expressed, L1 protein can self-assemble to form virus-like particle (VLP proteins) that are aggregates of approximately 72 L1 pentamers, similar to the virion coat. VLP protein can induce neutralizing antibodies in inoculated animals to protect experimental animals from subsequent attack by infectious viruses. Therefore, the VLP protein appears to be an excellent candidate for a papillomavirus vaccine. (Structure of Small Virus-like Particles Assembled from the L1 Protein of Human Papillomavirus 16 Chen, X. S., R. L. Garcea, Mol.Cell.5(3):557-567, 2000).

The term “reporter group” generally refers to the portion of the emission that produces detectable radiation, such as fluorescent or luminescent radiation, including but not limited to enzymes or radioisotopes. When the reporter group is an enzyme such as alkaline phosphatase, horseradish peroxidase or beta-d-galactosidase, a suitable substrate produces a color change upon reaction with the enzyme and the measurement of color intensity is quantified using a spectrophotometer. Or when the reporter group is a radioisotope, using a suitable gamma or beta ray detector. The strength of the reporter group is positively correlated to the amount of the substance to be tested in the test sample.

The present disclosure will be described in detail below by way of examples.

### Example 1: Construction and Screening Isolation of Antibody Library

1. Animal Immunity and Titer Detection

1.1 Animal Immunity

The HPV6 VLP protein (SEQ ID NO. 1, protein serial number: P69898& P06416) expressed in insect cell system was emulsified with Freund's complete adjuvant (Sigma Co., Art. No. F5881) and Freund's incomplete adjuvant (Sigma Co., Art. No.F5506) respectively to prepare Freund's complete adjuvant immunogen and Freund's incomplete adjuvant immunogen, wherein the volume ratio of HPV6 VLP(500 μg/animal) to Freund's complete adjuvant and Freund's incomplete adjuvant was 1:1.

Japanese white rabbits (2-2.2 kg, purchased from Beijing Shundong Culture Co., Ltd.) were immunized with Freund's complete adjuvant immunogen by subcutaneous multiple-point injection at the back. After the first immunization, animals were boosted with incomplete Freund's adjuvant immunogen at 2-week intervals in the same way and at the same dose. Blood was collected from the auricular vein on the 4th day after the fourth immunization to determine serum titer (for the procedures, see Example 1.2). The positive standard of serum titer was determined as (absorbance of antiserum-absorbance of blank)/(absorbance of negative control serum before immunization—absorbance of blank)>2.1. According to this standard, the spleen and bone marrow tissues were pooled to screen rabbit monoclonal antibodies for animals with serum titer up to 1:25000 one week after the last immunization.

1.2 Serological Test Titer

A proper amount of HPV6 VLP protein was diluted to 0.1 μg/mL with coating solution (0.05M Na₂CO₃, 0.05M NaHCO₃, pH 9.6, and sterile filtration through 0.2 μm); then 100 μL was added into each well of 96-well plate using a single-channel pipette; the samples were mixed evenly by tapping the plate, sealed with preservative film, and coated overnight at 4° C. The plate was washed once with washing solution (TBS containing 0.05% Tween20, pH 7.2-7.4) at 200 μL/well, and the enzyme-labeled plate was dried. The ELISA plate was blocked with blocking solution (washing solution containing 2% BSA) at 300 μL/well and blocked for 1 hour at room temperature. The plate was washed with washing solution at 300 μL/well twice and the ELISA plate was dried. The rabbit serum to be detected was subjected to gradient dilution with sample diluent, and the samples with the dilution ratios of 1:25000 and 1:125000 and the sample diluent were added at 100 μL/well, and the horseradish enzyme-labeled goat anti-rabbit IgG(H+L) detection antibody (IR, Art. No. 111-035-008) was added at 100 μL/well into a 96-well plate and performing for 2 hours at room temperature. The plate was washed three times with washing solution at 200 μL/well, and the enzyme-labeled plate was dried. The color developing solution was added at 200 μL/well (the substrate stock solution was diluted 1000 times with the substrate diluent, and 320 μL 0.75% H₂O₂ was added into the buffer after dilution for each liter, and used after uniform mixing) and placed at room temperature for 12 minutes. Stop the reaction by adding stop solution (2 M H₂SO₄) at 50 μL/well; detect by microplate reader: the wavelength for measurement is 450 nm.

2. Preparation and Screen of Phage Antibody Library

The spleen and bone marrow tissues of rabbits were RNA extracted with TriPure Isolation Reagent (source: Roche), and reversely transcribed with a reverse transcription kit (source: Invitrogen) to obtain cDNA, and the light-chain variable region sequence and the heavy-chain variable region sequence of the rabbit antibody were amplified by PCR (primer reference: Rader et al., 2000). The light-chain PCR primers are shown in Table 1 and the heavy-chain PCR primers are shown in Table 2, and the light-chain and heavy-chain variable region sequences encoding the rabbit antibody are spliced into nucleotide sequences encoding the scFv by an overlapping extension splicing PCR method, The variable region of light chains and heavy chains are connected by a linker TCTAGTGGTGGCGGTGGTTCGGGCGGTGGTGGAGGTGGTAGTTCTAGATCTTCC(e ncoding SSGGGGSGGGGGGSSRSS) (SEQ ID NO. 2), Then, the fragments were connected to a phage vector pComb3× (Sino Biological, Inc.) by restriction endonuclease SfiI (Fermentas), and the X-Blue competence (Sino Biological, Inc.) was electrically converted to construct a phage display scFv antibody library for immunized rabbits.

TABLE 1
Light chain PCR primers

| | | |
|---|---|---|
| SEQ ID NO. 27 | VKF1 | GAGCTCGTGMTGACCCAGACTCCA |
| SEQ ID NO. 28 | VKF2 | GAGCTCGATMTGACCCAGACTCCA |
| SEQ ID NO. 29 | VKF3 | GAGCTCGTGATGACCCAGACTGAA |
| SEQ ID NO. 30 | VKR1 | TAGGATCTCCAGCTCGGTCCC |
| SEQ ID NO. 31 | VKR2 | TTTGATTTCCACATTGGTGCC |
| SEQ ID NO. 32 | VKR3 | TTTGACSACCACCTCGGTCCC |

TABLE 2
Heavy chain PCR primers

| | | |
|---|---|---|
| SEQ ID NO. 33 | VHF1 | CAGTCGGTGGAGGAGTCCRGG |
| SEQ ID NO. 34 | VHF2 | CAGTCGGTGAAGGAGTCCGAG |
| SEQ ID NO. 35 | VHF3 | CAGTCGYTGGAGGAGTCCGGG |
| SEQ ID NO. 36 | VHF4 | CAGSAGCAGCTGRTGGAGTCCGG |
| SEQ ID NO. 37 | VHR1 | TGARGAGAYGGTGACCAGGGTGCC |

The HPV6 VLP protein was coated on an ELISA plate, and a phage library enriched with anti-HPV6 VLP positive antibodies was obtained by screening according to the procedure of phage antibody panning (Reference: Antibody Phase Display: Methods and Protocols, Philippa M. O'Brien, Humana Press).

Monoclonal bacteriophage was selected from the enriched library for expression, and the binding to HPV6 VLP protein was detected by ELISA method. The ELISA detection data are shown in Table 3. High-binding antibody clones specifically binding to HPV6 VLP protein were obtained by screening and sent to sequencing company for sequencing to obtain nucleotide sequence, wherein the nucleotide sequence of R003 scFv antibody is SEQ ID NO. 3, and the corresponding amino acid sequence is SEQ ID NO. 4.

TABLE 3
Detection data of HPV6 VLP monoclonal phage
expression by ELISA

| Coating: | HPV6 | | |
|---|---|---|---|
| Sample: | 2 μg/mL | PBS | CBS |
| 10fold dilution | OD450 | OD450 | OD450 |
| HPV6-R001 | 2.500 | 0.174 | 0.076 |
| HPV6-R003 | 2.501 | 0.482 | 0.223 |
| HPV6-R004 | 2.017 | 0.109 | 0.071 |
| HPV6-R015 | 1.942 | 0.276 | 0.151 |
| HPV6-R021 | 1.801 | 0.286 | 0.157 |
| HPV6-R022 | 1.683 | 0.345 | 0.265 |
| HPV6-R023 | 1.780 | 0.205 | 0.112 |
| HPV6-R025 | 1.085 | 0.225 | 0.131 |
| HPV6-R028 | 2.150 | 0.165 | 0.122 |
| HPV6-R029 | 1.524 | 0.174 | 0.106 |
| HPV6-R030 | 0.930 | 0.115 | 0.133 |
| HPV6-R031 | 1.260 | 0.161 | 0.149 |
| HPV6-R033 | 1.011 | 0.218 | 0.157 |
| HPV6-R041 | 1.422 | 0.169 | 0.133 |

The amino acid sequences of three CDRs in light chain and heavy chain of R003 scFv antibody were determined by referring to Kabat(Abhinandan and Martin 2008,Dondelinger, Filée et al. 2018) and IMGT number (Lefranc 2014) methods. See Table 4(SEQ ID NO. 5-10) for sequence information.

TABLE 4
Amino acid sequence of 3 CDRs in each of the
light and heavy chains of R003 scFv antibody

| | | |
|---|---|---|
| SEQ ID NO. 5 | Amino acid sequence | QASQSVYSNNLV |
| | of light chain CDR1 | |
| SEQ ID NO. 6 | Amino acid sequence | GASTLAS |
| | of light chain CDR2 | |
| SEQ ID NO. 7 | Amino acid sequence | GGYVSDSTDGTA |
| | of light chain CDR3 | |
| SEQ ID NO. 8 | Amino acid sequence | NYYMC |
| | of heavy chain CDR1 | |
| SEQ ID NO. 9 | Amino acid sequence | CIYTGSGRTYYA |
| | of heavy chain CDR2 | SWAKG |
| SEQ ID NO. 10 | Amino acid sequence | DYLGWRASNI |
| | of heavy chain CDR3 | |

### Example 2 Construction, Production and Purification of Antibody

The nucleotide sequence (SEQ ID NO. 11) of the heavy chain variable region of R003 scFv antibody (corresponding amino acid sequence is SEQ ID NO. 12) was obtained by PCR, and then the complete expression vector of the heavy chain nucleotide sequence (SEQ ID NO. 17) (corresponding amino acid sequence is SEQ ID NO. 18) was obtained by inserting it into the pSTEP2 vector with the nucleotide sequence of heavy chain signal peptide (SEQ ID NO. 13) (corresponding amino acid sequence is SEQ ID NO. 14) and the nucleotide sequence of rabbit IgG1 heavy chain constant region (SEQ ID NO. 15) (corresponding amino acid sequence is SEQ ID NO. 16) by ScaI and KpnI digestion.

The nucleotide sequence (SEQ ID NO. 19) of the light chain variable region of R003 scFv antibody was obtained by PCR, and the corresponding amino acid sequence was SEQ ID No. 20, and then the complete expression vector of the light chain nucleotide sequence (SEQ ID NO. 25) (corresponding amino acid sequence is SEQ ID NO. 26) was obtained by inserting it into the pSTEP2 vector with the nucleotide sequence of light chain signal peptide (SEQ ID NO. 21) (corresponding amino acid sequence is SEQ ID NO. 22) and the nucleotide sequence of rabbit kappa light chain constant region (SEQ ID NO. 23) (corresponding amino acid sequence is SEQ ID NO. 24) by ScaI and BamHI digestion. After the plasmid was extracted, it was transfected into HEK-293 cells for culture and expression for 7 days. The culture supernatant was purified by protein A purification column to obtain the high-purity antibody HPV6-R003.

TABLE 5
Primers of heavy chain variable region of
R003 scFv antibody obtained by PCR method

| | | |
|---|---|---|
| SEQ ID NO. 38 | F1 | ACCAGGGTGCTGAGTCAGCAGCAGCTGGAGGAG |
| SEQ ID NO. 39 | R1 | TGTGACCAGGGTACCTGGGCCCCA |

TABLE 6
Primers of light chain variable region of
R003 scFv antibody obtained by PCR method

| | | |
|---|---|---|
| SEQ ID NO. 40 | F2 | ACAGGAGTGCATAGTGAGCTCGTGAT |
| | | GACCCAGAC |
| SEQ ID NO. 41 | R2 | GGTGCAACTGGATCCCCTTTGACGAC |
| | | CACCTCGGT |

### Example 3 Biological Identification of Antibodies

1. Antibody Specificity Identification

1.1 Monoclonal Antibody HPV6-R003 does not Cross-React with Other Types of HPV VLP

Cross reactivity was detected by indirect ELISA method. The complete VLP protein of HPV6, HPV11, HPV16, HPV18, HPV31, HPV33, HPV35, HPV39, HPV45, HPV51, HPV52, HPV56, HPV58 and HPV59 can be diluted into 2 μg/mL respectively with phosphate buffer solution with pH of 7.2, and 100 μL/well coat enzyme labeling plate. The antibody to be examined was diluted to 10 ng/mL, and the horseradish peroxidase-labeled goat anti-rabbit Fc secondary antibody (Jackson, Art. No. 111035046) was used for coloration.

The sequence information of each coating protein and the specific identification results of HPV6-R003 antibody are shown in Table 7. The results show that the monoclonal antibody HPV6-R003 has good specificity, specifically binds to HPV6 VLP, and has no cross-reaction with HPV types 16, 11, 18, 31, 33, 35, 39, 45, 51, 52, 56, 58 and 59.

TABLE 7
Sequence information of coating protein
and specificity identification results of
HPV6-R003 antibody

| | | Protein | |
|---|---|---|---|
| Coating | Sequence | sequence | OD450- |
| protein | composition | number | blank |
| HPV6 | HPV6L1(1-469Y) | P69898 | 2.417 |
| | HPV33L1(474K-499K) | P06416 | |
| HPV11 | HPV11L1(1-470Y) | P04012 | 0.053 |
| | HPV33L1(474K-499K) | P06416 | |
| HPV16 | HPV-16L1 | P03101 | 0.048 |
| HPV18 | P18SHELL | Q80B70 | 0.045 |
| HPV31 | P31SHELL(1-475Y) | P17388 | 0.047 |
| | HPV33L1(474K-499K) | P06416 | |
| HPV33 | HPV33L1 | P06416 | 0.047 |
| HPV35 | HPV35L1(1-472L) | P27232 | 0.048 |
| | HPV33L1(474K-499K) | P06416 | |
| HPV39 | HPV39L1(1-469L) | P24838 | 0.047 |
| | HPV59L1(471L-508K) | Q81971 | |
| HPV45 | HPV45L1(1-478L) | P36741 | 0.061 |
| | HPV33L1(474K-499K) | P06416 | |
| HPV51 | HPV51L1 | P26536 | 0.041 |
| HPV52 | P52SHELL | Q05138 | 0.044 |
| HPV56 | HPV56L1 | P36743 | 0.042 |
| HPV58 | P58SHELL | P26535 | 0.044 |
| HPV59 | HPV59L1 | Q81971 | 0.041 |

1.2 Antibody Pairing Detection

Based on the results of antibody specificity and neutralization activity, HPV6-R003 with good specificity and strong neutralization activity was selected to establish a quantitative detection method for HPV6 VLP protein by using the double-antibody sandwich method.

HPV6-R003 was diluted to 2 μg/mL with phosphate buffer pH 7.2 and the ELISA plate was coated with 100 μL/well. HPV6 VLP protein with the concentration of 50 ng/mL was used as the standard substance, and after six consecutive 2-fold gradient dilutions, the samples were added into reaction wells with the volume of 100 μl/well. At the same time, the sample dilutions (0.1% BSA, 0.05% Tween20, 20 mM Tris, 150 mM NaCl, pH 7.2-7.4) were added to test as the blank control samples. Then, 1 μg/mL horseradish peroxidase-labeled HPV6-R003 was added into a volume of 100 μl/well to detect HPV6 VLP protein. After TMB substrate was added for coloration, the reaction was stopped under the action of acid, and the absorbance (OD value) at 450 nm was read. The OD value at 450 nm was positively correlated with the HPV6VLP protein in the sample. The standard curve was established based on the existing content of reference substances to calculate the content of HPV6VLP protein in the sample to be examined.

The OD values of the HPV6VLP_standard at 450 nm are shown in Table 8.

TABLE 8
Standard curve for quantitative detection of HPV6 VLP

| Concentration of HPV6-VLP | |
|---|---|
| (ng/mL) | Effective OD value |
| 0 | 0 |
| 0.78 | 0.027 |
| 1.56 | 0.043 |
| 3.12 | 0.102 |
| 6.25 | 0.207 |
| 12.50 | 0.439 |
| 25.00 | 0.921 |
| 50.00 | 1.836 |

Valid OD Value=OD_{Sample}−OD_{blank}

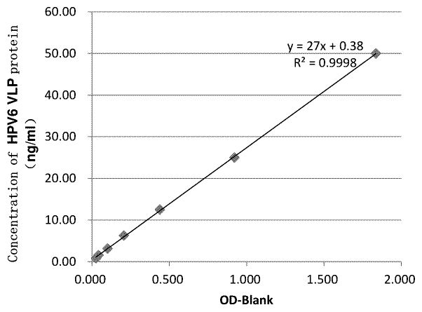

Thereby draw the standard curve, as shown in FIG. 1.

Performing cross-reaction detection on a mixture of 13 types of HPV (intact VLP proteins of HPV11, HPV16, HPV18, HPV31, HPV33, HPV35, HPV39, HPV45, HPV51, HPV52, HPV56, HPV58 and HPV59) except for HPV6 by using above detection method to evaluate the specificity of the detection method.

The mixtures of 13 HPV types other than HPV6 were diluted to 50 ng/mL (each subtype of HPV VLP in the mixture accounts for 1/13 respectively.) and tested by loading at 100 μl/well volume. At the same time, the sample dilutions (0.1% BSA, 0.05% Tween20, 20 mM Tris, 150 mM NaCl, pH 7.2-7.4) were loaded for test as the blank control sample. Three wells were repeated for reading the absorbance (OD value) at 450 nm.

When the mixtures of other 13 HPV types except HPV6 were detected by this method, the average OD450 value in the sample well was less than the average OD450 value in the blank control well plus 3 times of standard deviation, i.e., there was no cross-reaction signal when the mixtures of other 13 HPV types were detected by this method. The data are shown in

TABLE 9
Cross-reaction test results of the mixture of 13 HPV types

| | | OD450- | OD450- | OD450- | | AV + |
|---|---|---|---|---|---|---|
| Sample | | 1 | 2 | 3 | AVERAGE | 3SD |
| Mixture of | blank | 0.072 | 0.064 | 0.069 | 0.068 | 0.080 |
| 13 subtypes | | | | | | |
| (unit: ng/mL) | 50 | 0.071 | 0.075 | 0.081 | 0.076 | |

Cross-reaction testing of a mixture of 13 HPV types showed that the monoclonal antibody HPV6-R003 did not cross-react with HPV11, 16, 18, 31, 33, 35, 39, 45, 51, 52, 56, 58, and 59 in the mixture.

The immunogen HPV6 VLP protein sequence and HPV6-R003 antibody sequence are shown in Table 10.

TABLE 10
Immunogen HPV6 VLP protein sequences and HPV6-R003 antibody sequences

| SEQUENCE | | |
|---|---|---|
| NUMBER | NAME | SEQUENCE |
| SEQ ID NO. 1 | Amino acid | MWRPSDSTVYVPPPNPVSKVVATDAYVTRTNIFYHASSSRLLAVGHP |
| | sequence of | YFSIKRANKTVVPKVSGYQYRVFKVVLPDPNKFALPDSSLFDPTTQRL |
| | HPV6 VLP | VWACTGLEVGRGQPLGVGVSGHPFLNKYDDVENSGSGGNPGQDNR |
| | protein | VNVGMDYKQTQLCMVGCAPPLGEHWGKGKQCTNTPVQAGDCPPLE |
| | (P69898& | LITSVIQDGDMVDTGFGAMNFADLQTNKSDVPIDICGTTCKYPDYLQ |
| | P06416) | MAADPYGDRLFFFLRKEQMFARHFFNRAGEVGEPVPDTLIIKGSGNR |
| | | TSVGSSIYVNTPSGSLVSSEAQLFNKPYWLQKAQGHNNGICWGNQLF |
| | | VTVVDTTRSTNMTLCASVTTSSTYTNSDYKEYMRHVEEYDLQFIFQL |
| | | CSITLSAEVMAYIHTMNPSVLEDWNFGLSPPPNGTLEDTYRYVQSQAI |
| | | TCQKPTPEKEKPDPYKNLSFWEVNLKEKFSSELDQYPLGRKFLLQSG |
| | | YKAKPKLKRAAPTSTRTSSAKRKKVKK |
| SEQ ID NO. 2 | Nucleotide | TCTAGTGGTGGCGGTGGTTCGGGCGGTGGTGGAGGTGGTAGTTCT |
| | sequence of | AGATCTTCC |
| | Linker of rabbit | (The amino acid sequence is SSGGGGSGGGGGGSSRSS) |
| | antibody scFv | |
| | used in | |
| | constructing | |
| | phage antibody | |
| | library | |
| SEQ ID NO. 3 | Nucleotide | GAGCTCGTGATGACCCAGACTCCATCCTCCGTGTCTGCAGCTGTGG |
| | sequence of R003 | GAGGCACAGTCACCATCAATTGTCAGGCCAGTCAGAGTGTTTATA |
| | scFv | GTAACAACCTAGTCTGGTATCAGCAGAAACCAGGGCAGCCTCCCA |
| | | AGCTCCTGATCTATGGTGCATCCACTCTGGCATCTGGGGTCCCATC |
| | | GCGGTTCAAAGGCAGTGGATCTGGGACACAGTTCACTCTCACCAT |
| | | CAGCGATGTGGTGTGTGACGATGCTGCCACTTACTACTGTGGAGG |
| | | GTATGTTAGTGATAGTACTGATGGTACTGCTTTCGGCGGAGGGAC |
| | | CGAGGTGGTCGTCAAATCTAGTGGTGGCGGTGGTTCGGGCGGTGG |
| | | TGGAGGTGGTAGTTCTAGATCTTCCCAGCAGCAGCTGGAGGAGTC |
| | | CGGGGGAGGCCTGGTCAAGCCTGGAGGAACCCTGACACTCACCTG |
| | | TAAAGCCTCTGGAATCGACCTCAGTAACTACTACATGTGCTGGGTC |
| | | CGCCAGGCTCCAGGGAAGGGGCTGGAGTGGATCGGATGCATTTAT |
| | | ACTGGTAGTGGTCGCACTTACTACGCGAGCTGGGCGAAAGGCCGA |
| | | TTCACCATCTCCAAGGCCTCGTCGACCACGGTGACTCTGCAAATGA |
| | | CCAGTCTGACAGCCGCGGACACGGCCACCTATTTCTGTGCGAGAG |
| | | ATTATCTTGGTTGGCGTGCCAGTAACATCTGGGGCCCAGGTACCCT |
| | | GGTCACAGTGAGCTCT |
| SEQ ID NO. 4 | Amino acid | ELVMTQTPSSVSAAVGGTVTINCQASQSVYSNNLVWYQQKPGQPPK |
| | sequence of R003 | LLIYGASTLASGVPSRFKGSGSGTQFTLTISDVVCDDAATYYCGGYVS |
| | scFv | DSTDGTAFGGGTEVVVKSSGGGGSGGGGGGSSRSSQQQLEESGGGL |
| | | VKPGGTLTLTCKASGIDLSNYYMCWVRQAPGKGLEWIGCIYTGSGRT |
| | | YYASWAKGRFTISKASSTTVTLQMTSLTAADTATYFCARDYLGWRA |
| | | SNIWGPGTLVTVSS |
| SEQ ID NO. 5 | Amino acid | QASQSVYSNNLV |
| | sequence of | |
| | CDR1 of R003 | |
| | scFv light chain | |
| SEQ ID NO. 6 | Amino acid | GASTLAS |
| | sequence of | |
| | CDR2 of R003 | |
| | scFv light chain | |
| SEQ ID NO. 7 | Amino acid | GGYVSDSTDGTA |
| | sequence of | |
| | CDR3 of R003 | |
| | scFv light chain | |
| SEQ ID NO. 8 | Amino acid | NYYMC |
| | sequence of | |
| | CDR1 of R003 | |
| | scFv heavy chain | |
| SEQ ID NO. 9 | Amino acid | CIYTGSGRTYYASWAKG |
| | sequence of | |
| | CDR2 of R003 | |
| | scFv heavy chain | |
| SEQ ID | Amino acid | DYLGWRASNI |
| NO. 10 | sequence of | |
| | CDR3 of R003 | |
| | scFv heavy chain | |
| SEQ ID | Nucleotide | CAGCAGCAGCTGGAGGAGTCCGGGGGAGGCCTGGTCAAGCCTGG |
| NO. 11 | sequence of R003 | AGGAACCCTGACACTCACCTGTAAAGCCTCTGGAATCGACCTCAG |
| | heavy chain | TAACTACTACATGTGCTGGGTCCGCCAGGCTCCAGGGAAGGGGCT |
| | variable region | GGAGTGGATCGGATGCATTTATACTGGTAGTGGTCGCACTTACTAC |
| | | GCGAGCTGGGCGAAAGGCCGATTCACCATCTCCAAGGCCTCGTCG |
| | | ACCACGGTGACTCTGCAAATGACCAGTCTGACAGCCGCGGACACG |
| | | GCCACCTATTTCTGTGCGAGAGATTATCTTGGTTGGCGTGCCAGTA |
| | | ACATCTGGGGCCCAGGTACCCTGGTCACAGTGAGCTCT |
| SEQ ID | Amino acid | QQQLEESGGGLVKPGGTLTLTCKASGIDLSNYYMCWVRQAPGKGLE |
| NO. 12 | sequence of R003 | WIGCIYTGSGRTYYASWAKGRFTISKASSTTVTLQMTSLTAADTATY |
| | heavy chain | FCARDYLGWRASNIWGPGTLVTVSS |
| | variable region | |
| SEQ ID | Nucleotide | ATGGGCTGGTCCCTGATTCTGCTGTTCCTGGTGGCTGTGGCTACCA |
| NO. 13 | sequence of R003 | GGGTGCTGAGT |
| | heavy chain | |
| | signal peptide | |
| SEQ ID | Amino acid | MGWSLILLFLVAVATRVLS |
| NO. 14 | sequence of R003 | |
| | heavy chain | |
| | signal peptide | |
| SEQ ID | Nucleotide | GGTCAACCTAAGGCTCCGTCAGTCTTCCCACTGGCCCCCTGCTGCG |
| NO. 15 | sequence of R003 | GGGACACACCCAGCTCCACGGTGACCCTGGGCTGCCTGGTCAAAG |
| | heavy chain | GCTACCTCCCGGAGCCAGTGACCGTGACCTGGAACTCGGGCACCC |
| | constant region | TCACCAATGGGGTACGCACCTTCCCGTCCGTCCGGCAGTCCTCAGG |
| | | CCTCTACTCGCTGAGCAGCGTGGTGAGCGTGACCTCAAGCAGCCA |
| | | GCCCGTCACCTGCAACGTGGCCCACCCAGCCACCAACACCAAAGT |
| | | GGACAAGACCGTTGCGCCCTCGACATGCAGCAAGCCCACGTGCCC |
| | | ACCCCCTGAACTCCTGGGGGGACCGTCTGTCTTCATCTTCCCCCCA |
| | | AAACCCAAGGACACCCTCATGATCTCACGCACCCCCGAGGTCACA |
| | | TGCGTGGTGGTGGACGTGAGCCAGGATGACCCCGAGGTGCAGTTC |
| | | ACATGGTACATAAACAACGAGCAGGTGCGCACCGCCCGGCCGCCG |
| | | CTACGGGAGCAGCAGTTCAACAGCACGATCCGCGTGGTCAGCACC |
| | | CTCCCCATCGCGCACCAGGACTGGCTGAGGGGCAAGGAGTTCAAG |
| | | TGCAAAGTCCACAACAAGGCACTCCCGGCCCCCATCGAGAAAACC |
| | | ATCTCCAAAGCCAGAGGGCAGCCCCTGGAGCCGAAGGTCTACACC |
| | | ATGGGCCCTCCCCGGGAGGAGCTGAGCAGCAGGTCGGTCAGCCTG |
| | | ACCTGCATGATCAACGGCTTCTACCCTTCCGACATCTCGGTGGAGT |
| | | GGGAGAAGAACGGGAAGGCAGAGGACAACTACAAGACCACGCCG |
| | | GCCGTGCTGGACAGCGACGGCTCCTACTTCCTCTACAGCAAGCTCT |
| | | CAGTGCCCACGAGTGAGTGGCAGCGGGGCGACGTCTTCACCTGCT |
| | | CCGTGATGCACGAGGCCTTGCACAACCACTACACGCAGAAGTCCA |
| | | TCTCCCGCTCTCCGGGTAAATAA |
| SEQ ID | Amino acid | GQPKAPSVFPLAPCCGDTPSSTVTLGCLVKGYLPEPVTVTWNSGTLT |
| NO. 16 | sequence of R003 | NGVRTFPSVRQSSGLYSLSSVVSVTSSSQPVTCNVAHPATNTKVDKT |
| | heavy chain | VAPSTCSKPTCPPPELLGGPSVFIFPPKPKDTLMISRTPEVTCVVVDVS |
| | constant region | QDDPEVQFTWYINNEQVRTARPPLREQQFNSTIRVVSTLPIAHQDWLR |
| | | GKEFKCKVHNKALPAPIEKTISKARGQPLEPKVYTMGPPREELSSRSV |
| | | SLTCMINGFYPSDISVEWEKNGKAEDNYKTTPAVLDSDGSYFLYSKL |
| | | SVPTSEWQRGDVFTCSVMHEALHNHYTQKSISRSPGK |
| SEQ ID | Nucleotide | ATGGGCTGGTCCCTGATTCTGCTGTTCCTGGTGGCTGTGGCTACCA |
| NO. 17 | sequence of R003 | GGGTGCTGAGTCAGCAGCAGCTGGAGGAGTCCGGGGGAGGCCTG |
| | heavy chain | GTCAAGCCTGGAGGAACCCTGACACTCACCTGTAAAGCCTCTGGA |
| | | ATCGACCTCAGTAACTACTACATGTGCTGGGTCCGCCAGGCTCCA |
| | | GGGAAGGGGCTGGAGTGGATCGGATGCATTTATACTGGTAGTGGT |
| | | CGCACTTACTACGCGAGCTGGGCGAAAGGCCGATTCACCATCTCC |
| | | AAGGCCTCGTCGACCACGGTGACTCTGCAAATGACCAGTCTGACA |
| | | GCCGCGGACACGGCCACCTATTTCTGTGCGAGAGATTATCTTGGTT |
| | | GGCGTGCCAGTAACATCTGGGGCCCAGGTACCCTGGTCACAGTGA |
| | | GCTCTGGTCAACCTAAGGCTCCGTCAGTCTTCCCACTGGCCCCCTG |
| | | CTGCGGGGACACACCCAGCTCCACGGTGACCCTGGGCTGCCTGGT |
| | | CAAAGGCTACCTCCCGGAGCCAGTGACCGTGACCTGGAACTCGGG |
| | | CACCCTCACCAATGGGGTACGCACCTTCCCGTCCGTCCGGCAGTCC |
| | | TCAGGCCTCTACTCGCTGAGCAGCGTGGTGAGCGTGACCTCAAGC |
| | | AGCCAGCCCGTCACCTGCAACGTGGCCCACCCAGCCACCAACACC |
| | | AAAGTGGACAAGACCGTTGCGCCCTCGACATGCAGCAAGCCCACG |
| | | TGCCCACCCCCTGAACTCCTGGGGGGACCGTCTGTCTTCATCTTCC |
| | | CCCCAAAACCCAAGGACACCCTCATGATCTCACGCACCCCCGAGG |
| | | TCACATGCGTGGTGGTGGACGTGAGCCAGGATGACCCCGAGGTGC |
| | | AGTTCACATGGTACATAAACAACGAGCAGGTGCGCACCGCCCGGC |
| | | CGCCGCTACGGGAGCAGCAGTTCAACAGCACGATCCGCGTGGTCA |
| | | GCACCCTCCCCATCGCGCACCAGGACTGGCTGAGGGGCAAGGAGT |
| | | TCAAGTGCAAAGTCCACAACAAGGCACTCCCGGCCCCCATCGAGA |
| | | AAACCATCTCCAAAGCCAGAGGGCAGCCCCTGGAGCCGAAGGTCT |
| | | ACACCATGGGCCCTCCCCGGGAGGAGCTGAGCAGCAGGTCGGTCA |
| | | GCCTGACCTGCATGATCAACGGCTTCTACCCTTCCGACATCTCGGT |
| | | GGAGTGGGAGAAGAACGGGAAGGCAGAGGACAACTACAAGACCA |
| | | CGCCGGCCGTGCTGGACAGCGACGGCTCCTACTTCCTCTACAGCA |
| | | AGCTCTCAGTGCCCACGAGTGAGTGGCAGCGGGGCGACGTCTTCA |
| | | CCTGCTCCGTGATGCACGAGGCCTTGCACAACCACTACACGCAGA |
| | | AGTCCATCTCCCGCTCTCCGGGTAAATAA |
| SEQ ID | Amino acid | MGWSLILLFLVAVATRVLSQQQLEESGGGLVKPGGTLTLTCKASGID |
| NO. 18 | sequence of R003 | LSNYYMCWVRQAPGKGLEWIGCIYTGSGRTYYASWAKGRFTISKAS |
| | heavy chain | STTVTLQMTSLTAADTATYFCARDYLGWRASNIWGPGTLVTVSSGQP |
| | | KAPSVFPLAPCCGDTPSSTVTLGCLVKGYLPEPVTVTWNSGTLTNGV |
| | | RTFPSVRQSSGLYSLSSVVSVTSSSQPVTCNVAHPATNTKVDKTVAPS |
| | | TCSKPTCPPPELLGGPSVFIFPPKPKDTLMISRTPEVTCVVVDVSQDDP |
| | | EVQFTWYINNEQVRTARPPLREQQFNSTIRVVSTLPIAHQDWLRGKEF |
| | | KCKVHNKALPAPIEKTISKARGQPLEPKVYTMGPPREELSSRSVSLTC |
| | | MINGFYPSDISVEWEKNGKAEDNYKTTPAVLDSDGSYFLYSKLSVPT |
| | | SEWQRGDVFTCSVMHEALHNHYTQKSISRSPGK |
| SEQ ID | Nucleotide | GAGCTCGTGATGACCCAGACTCCATCCTCCGTGTCTGCAGCTGTGG |
| NO. 19 | sequence of R003 | GAGGCACAGTCACCATCAATTGTCAGGCCAGTCAGAGTGTTTATA |
| | light chain | GTAACAACCTAGTCTGGTATCAGCAGAAACCAGGGCAGCCTCCCA |
| | variable region | AGCTCCTGATCTATGGTGCATCCACTCTGGCATCTGGGGTCCCATC |
| | | GCGGTTCAAAGGCAGTGGATCTGGGACACAGTTCACTCTCACCAT |
| | | CAGCGATGTGGTGTGTGACGATGCTGCCACTTACTACTGTGGAGG |
| | | GTATGTTAGTGATAGTACTGATGGTACTGCTTTCGGCGGAGGGAC |
| | | CGAGGTGGTCGTCAAA |
| SEQ ID | Amino acid | ELVMTQTPSSVSAAVGGTVTINCQASQSVYSNNLVWYQQKPGQPPK |
| NO. 20 | sequence of R003 | LLIYGASTLASGVPSRFKGSGSGTQFTLTISDVVCDDAATYYCGGYVS |
| | light chain | DSTDGTAFGGGTEVVVK |
| | variable region | |
| SEQ ID | Nucleotide | ATGGGCTGGTCCTGTATCATCCTGTTCCTGGTGGCTACAGCCACAG |
| NO. 21 | sequence of R003 | GAGTGCATAGT |
| | light chain signal | |
| | peptide | |
| SEQ ID | Amino acid | MGWSCIILFLVATATGVHS |
| NO. 22 | sequence of R003 | |
| | light chain signal | |
| | peptide | |
| SEQ ID | Nucleotide | GGGGATCCAGTTGCACCTACTGTCCTCATCTTCCCACCAGCTGCTG |
| NO. 23 | sequence of R003 | ATCAGGTGGCAACTGGAACAGTCACCATCGTGTGTGTGGCGAATA |
| | light chain | AATACTTTCCCGATGTCACCGTCACCTGGGAGGTGGATGGCACCA |
| | constant region | CCCAAACAACTGGCATCGAGAACAGTAAAACACCGCAGAATTCTG |
| | | CAGATTGTACCTACAACCTCAGCAGCACTCTGACACTGACCAGCA |
| | | CACAGTACAACAGCCACAAAGAGTACACCTGCAAGGTGACCCAG |
| | | GGCACGACCTCAGTCGTCCAGAGCTTCAATAGGGGTGACTGTTAA |
| SEQ ID | Amino acid | GDPVAPTVLIFPPAADQVATGTVTIVCVANKYFPDVTVTWEVDGTTQ |
| NO. 24 | sequence of R003 | TTGIENSKTPQNSADCTYNLSSTLTLTSTQYNSHKEYTCKVTQGTTSV |
| | light chain | VQSFNRGDC |
| | constant region | |
| SEQ ID | Nucleotide | ATGGGCTGGTCCTGTATCATCCTGTTCCTGGTGGCTACAGCCACAG |
| NO. 25 | sequence of R003 | GAGTGCATAGTGAGCTCGTGATGACCCAGACTCCATCCTCCGTGTC |
| | light chain | TGCAGCTGTGGGAGGCACAGTCACCATCAATTGTCAGGCCAGTCA |
| | | GAGTGTTTATAGTAACAACCTAGTCTGGTATCAGCAGAAACCAGG |
| | | GCAGCCTCCCAAGCTCCTGATCTATGGTGCATCCACTCTGGCATCT |
| | | GGGGTCCCATCGCGGTTCAAAGGCAGTGGATCTGGGACACAGTTC |
| | | ACTCTCACCATCAGCGATGTGGTGTGTGACGATGCTGCCACTTACT |
| | | ACTGTGGAGGGTATGTTAGTGATAGTACTGATGGTACTGCTTTCGG |
| | | CGGAGGGACCGAGGTGGTCGTCAAAGGGGATCCAGTTGCACCTAC |
| | | TGTCCTCATCTTCCCACCAGCTGCTGATCAGGTGGCAACTGGAACA |
| | | GTCACCATCGTGTGTGTGGCGAATAAATACTTTCCCGATGTCACCG |
| | | TCACCTGGGAGGTGGATGGCACCACCCAAACAACTGGCATCGAGA |
| | | ACAGTAAAACACCGCAGAATTCTGCAGATTGTACCTACAACCTCA |
| | | GCAGCACTCTGACACTGACCAGCACACAGTACAACAGCCACAAAG |
| | | AGTACACCTGCAAGGTGACCCAGGGCACGACCTCAGTCGTCCAGA |
| | | GCTTCAATAGGGGTGACTGTTAA |
| SEQ ID | Amino acid | MGWSCIILFLVATATGVHSELVMTQTPSSVSAAVGGTVTINCQASQS |
| NO. 26 | sequence of R003 | VYSNNLVWYQQKPGQPPKLLIYGASTLASGVPSRFKGSGSGTQFTLTI |
| | light chain | SDVVCDDAATYYCGGYVSDSTDGTAFGGGTEVVVKGDPVAPTVLIF |
| | | PPAADQVATGTVTIVCVANKYFPDVTVTWEVDGTTQTTGIENSKTPQ |
| | | NSADCTYNLSSTLTLTSTQYNSHKEYTCKVTQGTTSVVQSFNRGDC |

The preferred embodiments of the present disclosure have been described above in detail, but the present disclosure is not limited thereto. Within the scope of the technical concept of the present disclosure, various simple modifications can be made to the technical scheme of the present disclosure, including the combination of various technical features in any other suitable manner, and these simple modifications and combinations can also be regarded as the disclosure of the present disclosure, and are within the protection scope of the present disclosure.

What is claimed is:

1. An anti-HPV6 L1 protein antibody or an antigen-binding fragment thereof comprising a light chain variable region or part thereof and/or a heavy chain variable region or part thereof,
wherein the light chain variable region or part thereof comprises one or more of a light chain CDR1 having an amino acid sequence of SEQ ID NO:5, a light chain CDR2 having an amino acid sequence of SEQ ID NO:6, and a light chain CDR3 having an amino acid sequence of SEQ ID NO:7; and
the heavy chain variable region or part thereof comprises one or more of a heavy chain CDR1 having an amino acid sequence of SEQ ID NO:8, a heavy chain CDR2 having an amino acid sequence of SEQ ID NO:9, and a heavy chain CDR3 having an amino acid sequence of SEQ ID NO:10.

2. The anti-HPV6 L1 protein antibody or antigen-binding fragment thereof of claim 1, wherein the antibody or antigen-binding fragment thereof comprises an amino acid sequence having at least 90%, 92%, 95%, 98% or 100% sequence identity to the amino acid sequence SEQ ID NO:20 of the light chain variable region of an anti-HPV6 L1 protein antibody, and/or
an amino acid sequence have at least 90%, 92%, 95%, 98% or 100% sequence identity to the amino acid sequence SEQ ID NO:12 of the heavy chain variable region of an anti-HPV6 L1 protein antibody.

3. The anti-HPV6 L1 protein antibody or antigen-binding fragment thereof of claim 1, wherein the antibody further comprises a light chain constant region and a heavy chain constant region.

4. The anti-HPV6 L1 protein antibody or antigen-binding fragment thereof of claim 2, wherein the antibody further comprises a light chain constant region and a heavy chain constant region.

5. The anti-HPV6 L1 protein antibody or antigen-binding fragment thereof of claim 3, wherein the light chain constant region is an amino acid sequence have at least 90%, 92%, 95%, 98% or 100% sequence identity to the amino acid sequence SEQ ID NO:24 of the light chain constant region of an anti-HPV6 L1 protein antibody, and/or
the heavy chain constant region is an amino acid sequence have at least 90%, 92%, 95%, 98% or 100% sequence identity to the amino acid sequence SEQ ID NO:16 of the heavy chain constant region of an anti-HPV6 L1 protein antibody.

6. The anti-HPV6 L1 protein antibody or antigen-binding fragment thereof of claim 4, wherein the light chain constant region is an amino acid sequence have at least 90%, 92%, 95%, 98% or 100% sequence identity to the amino acid sequence SEQ ID NO:24 of the light chain constant region of an anti-HPV6 L1 protein antibody, and/or
the heavy chain constant region is an amino acid sequence have at least 90%, 92%, 95%, 98% or 100% sequence identity to the amino acid sequence SEQ ID NO:16 of the heavy chain constant region of an anti-HPV6 L1 protein antibody.

7. The anti-HPV6 L1 protein antibody or antigen-binding fragment thereof of claim 1, wherein the antibody is a monoclonal antibody.

8. The anti-HPV6 L1 protein antibody or antigen-binding fragment thereof of claim 2, wherein the antibody is a monoclonal antibody.

9. The anti-HPV6 L1 protein antibody or antigen-binding fragment thereof of claim 1, wherein the antibody specifically binds to the HPV6 L1 antigen and does not cross-react with other types of HPV L1 antigens.

10. The anti-HPV6 L1 protein antibody or antigen-binding fragment thereof of claim 2, wherein the antibody specifically binds to the HPV6 L1 antigen and does not cross-react with other types of HPV L1 antigens.

11. The anti-HPV6 L1 protein antibody or antigen-binding fragment thereof of claim 9, wherein the other HPV types are HPV types 11, 16, 18, 31, 33, 35, 39, 45, 51, 52, 56, 58 and 59.

12. The anti-HPV6 L1 protein antibody or antigen-binding fragment thereof of claim 10, wherein the other HPV types are HPV types 11, 16, 18, 31, 33, 35, 39, 45, 51, 52, 56, 58 and 59.

13. The anti-HPV6 L1 protein antibody or antigen-binding fragment thereof of claim 1, wherein the antigen-binding fragment is in the form of Fv, Fab, Fab′, Fab′-SH, F(ab′)2, Fd fragment, Fd′ fragment, single chain antibody molecule or single-domain antibody.

14. The anti-HPV6 L1 protein antibody or antigen-binding fragment thereof of claim 2, wherein the antigen-binding fragment is in the form of Fv, Fab, Fab′, Fab′-SH, F(ab′)2, Fd fragment, Fd′ fragment, single chain antibody molecule or single-domain antibody.

15. The anti-HPV6 L1 protein antibody or antigen-binding fragment thereof of claim 13, wherein the single-chain antibody molecule is an scFv, a di-scFv, a tri-scFv, a bispecific antibody, or an scFab.

16. The anti-HPV6 L1 protein antibody or antigen-binding fragment thereof of claim 14, wherein the single-chain antibody molecule is an scFv, a di-scFv, a tri-scFv, a bispecific antibody, or an scFab.

17. A nucleic acid comprising: a nucleotide sequence encoding the anti-HPV6 L1 protein antibody or antigen-binding fragment thereof of claim 1.

18. A method for detecting the presence of HPV6 L1 protein in a sample comprising:
a) coating the anti-HPV6 L1 protein antibody or antigen-binding fragment thereof of claim 1 onto an assay surface;
b) contacting said assay surface with the sample to be tested for a period of time sufficient to allow binding, and then washing said assay surface;
c) contacting the assay surface with the anti-HPV6 L1 protein antibody or antigen-binding fragment thereof of claim 1 conjugated to a reporter group for a period of time sufficient to allow binding of the conjugated antibody, and then washing the assay surface; and
d) detecting the signal of the reporter group.

19. The method of claim 18, wherein the antibodies of claim 1 in steps (a) and (c) are the same or different.

20. The method of claim 19, wherein the reporter group is horseradish peroxidase.
